(19) United States
(12) Patent Application Publication
(10) Pub. No.: US 2023/0248462 A1
(43) Pub. Date: Aug. 10, 2023

(54) CLITORIS SHIELD

(71) Applicant: Michelle Denise Cooper, Gypsum, CO (US)

(72) Inventor: Michelle Denise Cooper, Gypsum, CO (US)

(21) Appl. No.: 18/056,052

(22) Filed: Nov. 16, 2022

Related U.S. Application Data
(60) Provisional application No. 63/308,845, filed on Feb. 10, 2022.

(51) Int. Cl. A61B 90/00; A61B 18/20

(52) U.S. Cl. CPC A61B 90/04; A61B 18/203; A61B 2090/0436; A61B 2090/049; A61B 2017/00752

(57) ABSTRACT

A clitoris shield has an oval body with a convex surface and a concave surface, a rectangular laser guide extending longitudinally from the oval body; and an arcuate laser guide extending radially from the convex surface. The clitoris shield is made of a material having a color that disperses, reflects, and/or blocks laser energy. The arcuate laser guard has a longitudinal axis parallel to the longitudinal axis of the oval body and the rectangular laser guide. The concave surface fits a clitoris. The clitoris shield prevents a woman from getting burned on her clitoris during laser hair removal, avoiding pain and permanent damage to the clitoris.

### CROSS-REFERENCE TO RELATED APPLICATION

This application claims the benefit of priority of U.S. provisional application No. 63/308,845, filed Feb. 10, 2022, the contents of which are herein incorporated by reference.

### BACKGROUND OF THE INVENTION

The present invention relates to laser hair removal and, more particularly, to a clitoris shield.

When a woman gets a Brazilian laser treatment, the laser technician must get very close to her clitoris to remove the hair within the labia area. In the process, she may unintentionally burn her client's clitoris, which is extremely painful and can cause permanent damage. Laser Technicians do not currently use anything to protect their clients from clitoris laser burns. Yet many clients claim they have been burned on their clitoris by their laser technician and have been caused pain or damage to the clitoris area.

No currently available device protects a clitoris during laser hair removal. A commercially available plastic device suitable for waxing is colored and cannot be used for laser hair removal because it will burn and/or be melted by the laser, causing an additional hazard.

As can be seen, there is a need for a nonflammable device that protects the clitoris that will not melt during laser hair removal.

### SUMMARY OF THE INVENTION

In one aspect of the present invention, a clitoris shield comprises an oval body having a convex surface and a concave surface and formed of a material having a color operative to disperse, reflect, and/or block laser energy; a rectangular laser guide extending longitudinally from the oval body; and an arcuate laser guide extending radially from the convex surface and having a longitudinal axis parallel to a longitudinal axis of the oval body and the rectangular laser guide. The concave surface is dimensioned to accommodate a clitoris.

The inventive device prevents a woman from getting burned on her clitoris by the laser, avoiding pain and permanent damage to the clitoris.

These and other features, aspects and advantages of the present invention will become better understood with reference to the following drawings, description, and claims.

### BRIEF DESCRIPTION OF THE DRAWINGS

FIG. 1 is a top perspective view of a clitoris shield according to an embodiment of the present invention;

FIG. 2 is a bottom perspective view thereof;

FIG. 3 is a top plan view thereof;

FIG. 4 is a bottom plan view thereof;

FIG. 5 is a sectional view thereof, taken along line 5-5 in FIG. 3; and

FIG. 6 is a schematic view thereof, shown in use.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a clear and/or white plastic clitoris shield. The clear or white plastic device does not burn or melt upon contact by the laser. The shield may be placed on or cupped over a woman's clitoris to prevent her from being burned by the laser during a Brazilian laser procedure. It enables the laser technician to move her laser around the clitoris without the woman getting burned.

The shape and dimensions of the device are not particularly limited. For example, the clitoris shield may have an oval shape about 1 to 3 inches wide and about 2 to 4 inches long with a straight edge 1 to 2 inches long extending from the oval body and a heart-shaped handle edge extending from a convex surface of the oval body at a 90° angle from the plane containing the oval shape.

The material of manufacture is not particularly limited, provided it is a sturdy and strong, yet soft (on the side edge), plastic that is clear and/or white. For example, the device may be made of HDPE or PP plastic material.

The method of manufacture is not particularly limited. For example, the device may be made by a plastic thermoformer.

The inventive device may be used by a laser technician by placing it over a woman's clitoris before using a laser to do Brazilian hair removal. The device may be reversed, turned around, and/or flipped 90° to serve as a guide for the laser technician to make a landing strip or heart shape using the laser. The inventive shield may also be used by a tattoo artist and in the medical industry.

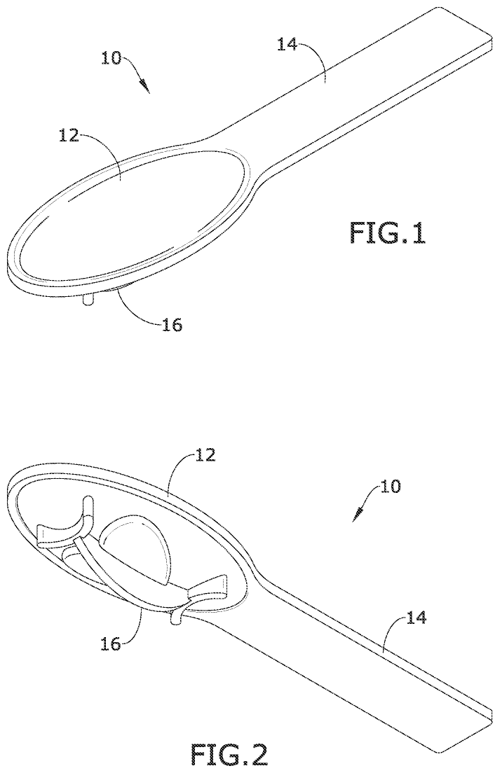

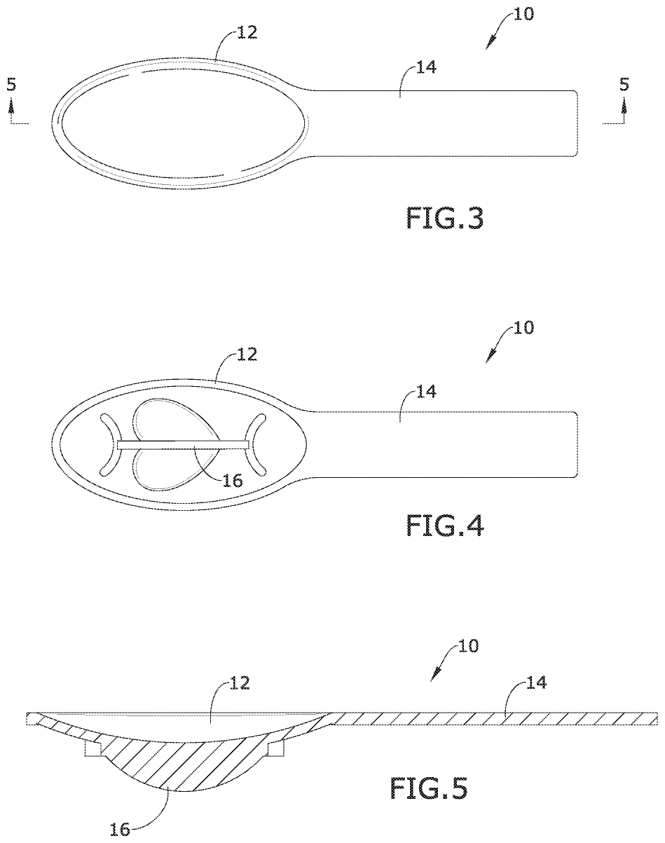

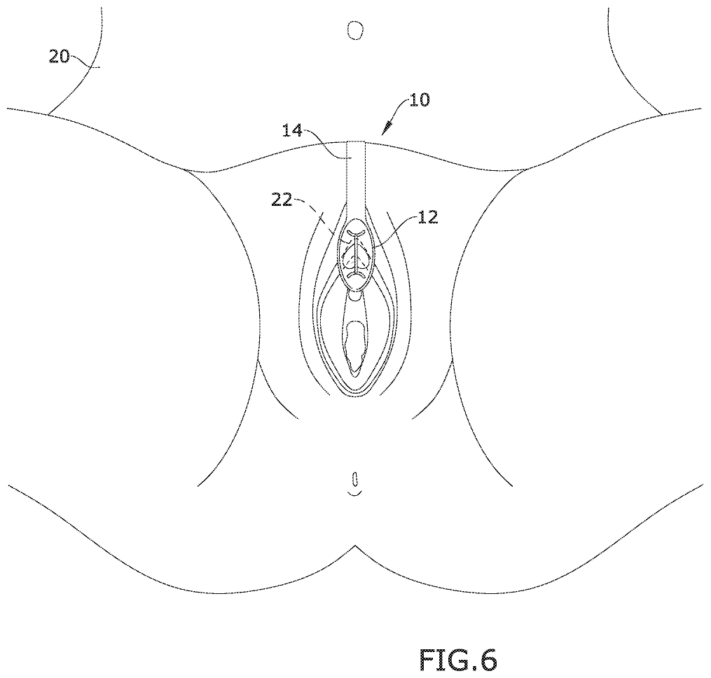

Referring to FIGS. 1 through 6, a clitoris shield 10 is shown. FIG. 1 illustrates a concave surface of an oval shaped paddle 12 with a straight edge or handle 14 extending therefrom. FIG. 2 illustrates the convex surface of the paddle 12 with a laser guide 16 extending therefrom. The plane through the laser guide 16 is positioned at a 90° angle from the plane through the straight edge 14, as shown in FIGS. 3-5. FIG. 6 shows how the clitoris shield 10 may be used to protect a user's 20 clitoris 22 during laser hair removal.

It should be understood, of course, that the foregoing relates to exemplary embodiments of the invention and that modifications may be made without departing from the spirit and scope of the invention as set forth in the following claims.

What is claimed is:

1. A clitoris shield comprising:
an oval body having a convex surface and a concave surface and formed of a material having a color operative to disperse, reflect, and/or block laser energy;
a rectangular laser guide extending longitudinally from the oval body; and
an arcuate laser guide extending radially from the convex surface and having a longitudinal axis parallel to a longitudinal axis of the oval body and the rectangular laser guide;
wherein the concave surface is dimensioned to accommodate a clitoris.

2. The clitoris shield of claim 1, wherein the oval body is about 1 to 3 inches wide and about 2 to 4 inches long.

3. The clitoris shield of claim 1, wherein the rectangular laser guide is about 1 to 2 inches long.

4. The clitoris shield of claim 1, comprising a clear and/or white plastic.

5. The clitoris shield of claim 4, wherein the plastic is polypropylene or high-density polyethylene.
